(19) United States
(12) Plant Patent Application Publication
(10) Pub. No.: US 2013/0340136 P1
(43) Pub. Date: Dec. 19, 2013

(54) Strawberry plants named 'Aurora', 'Crystalina', Fiona', and 'Marina'

(50) ;

(71) Applicant: Sweet Darling Sales, Inc., Aptos, CA (US)

(72) Inventor: John Larse, Aptos, CA (US)

(73) Assignee: Sweet Darling Sales, Inc., Aptos, CA (US)

(21) Appl. No.: 13/986,676

(22) Filed: May 23, 2013

Related U.S. Application Data
(60) Provisional application No. 61/650,798, filed on May 23, 2012.

(51) Int. Cl. A01H 5/00

(52) U.S. Cl. PLT/208

(57) ABSTRACT

The present invention relates to new and distinct day-neutral strawberry varieties designated as ‘AURORA’ (a.k.a. ‘107750’), ‘CRYSTALINA’ (a.k.a. ‘107936’, or ‘CRYSTAL’ previously), ‘FIONA’ (a.k.a. ‘108230’), and ‘MARINA’ (a.k.a. ‘108074’).

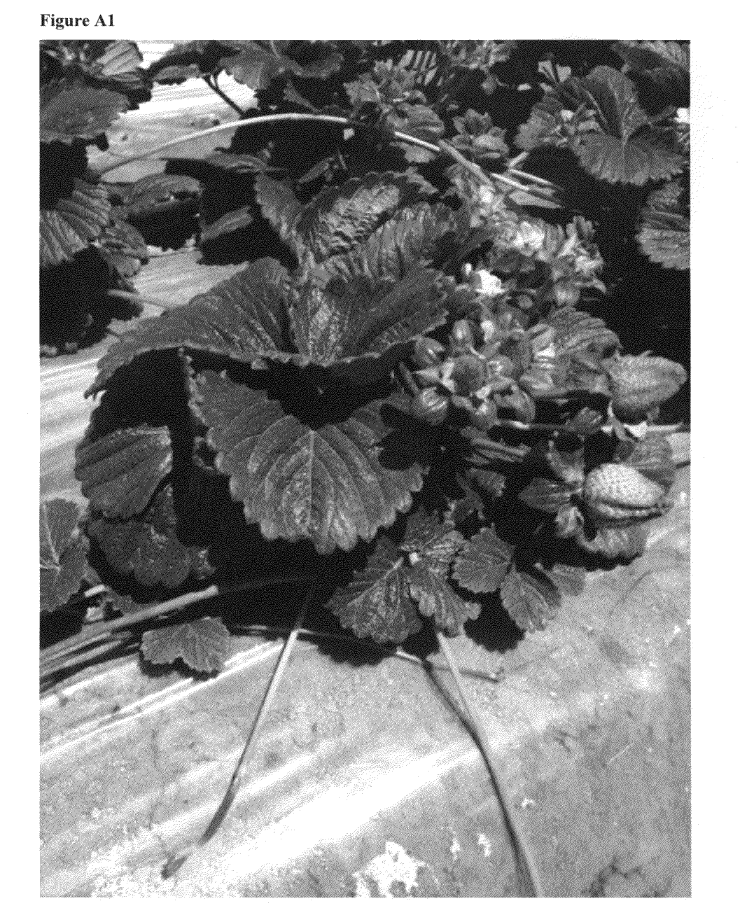

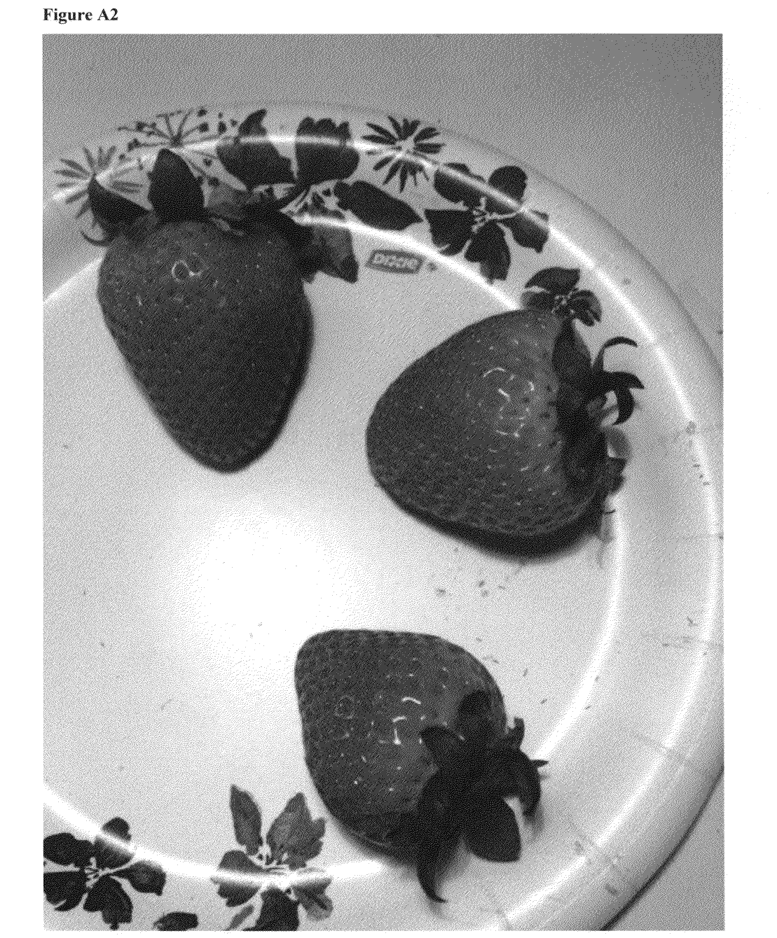

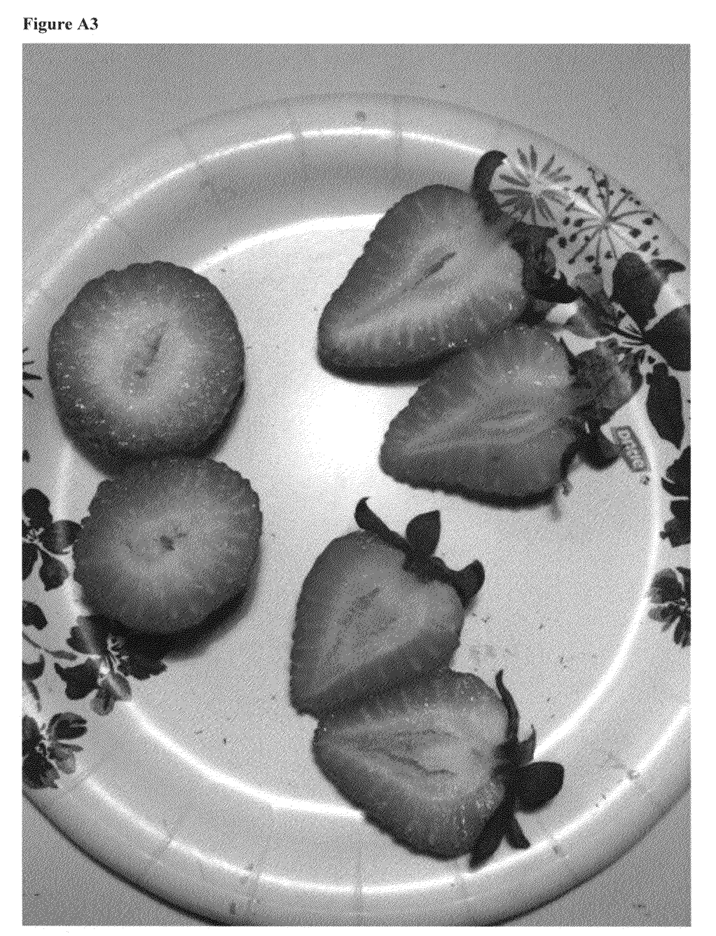

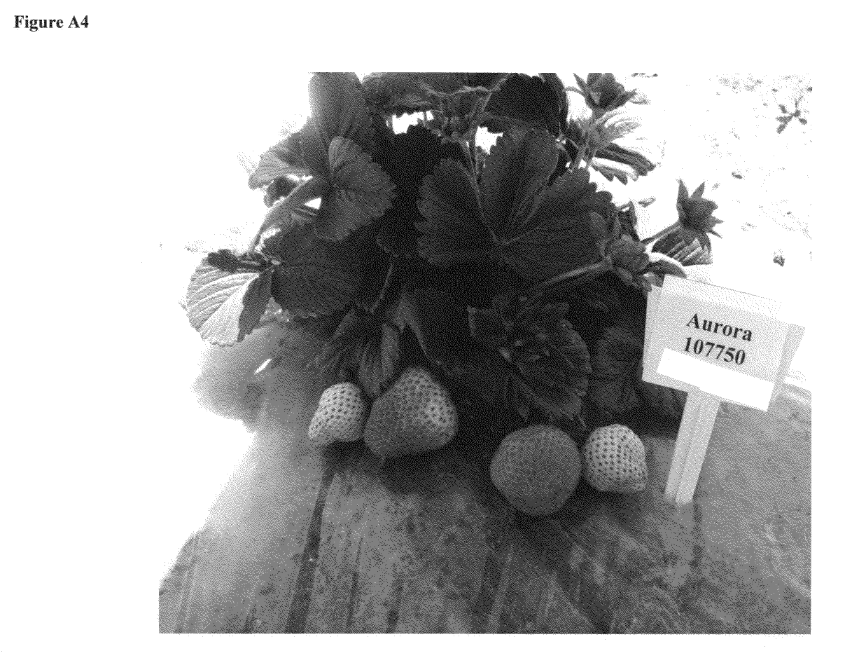

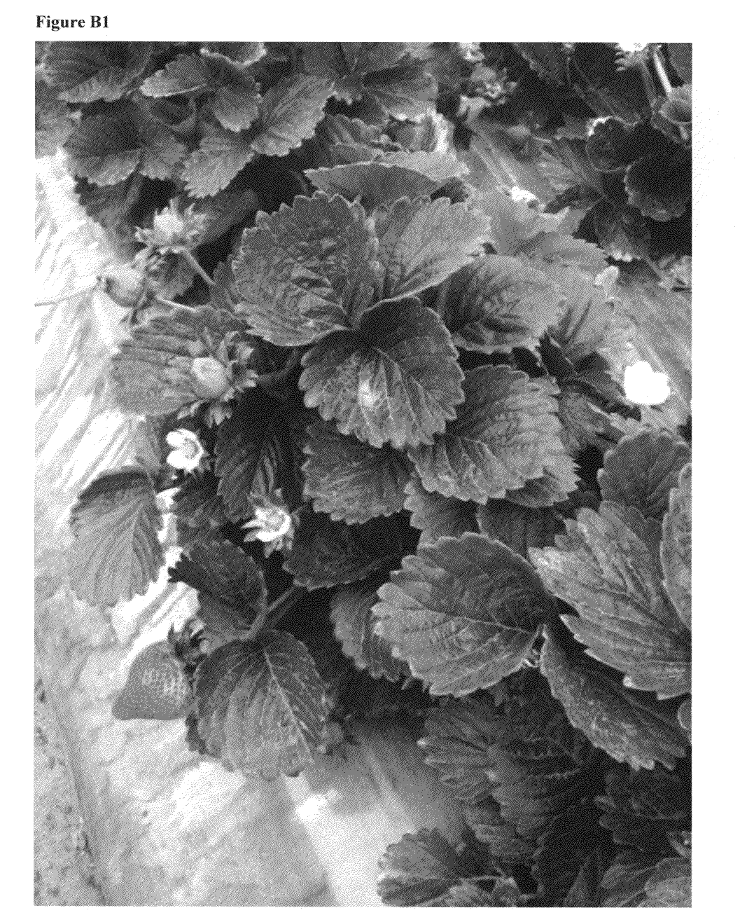

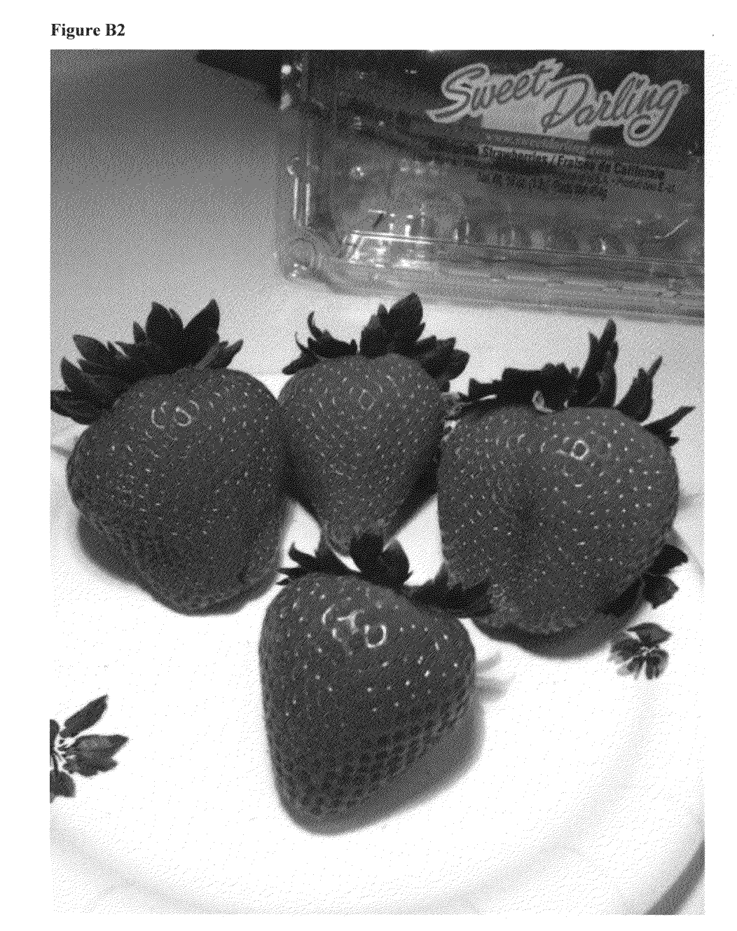

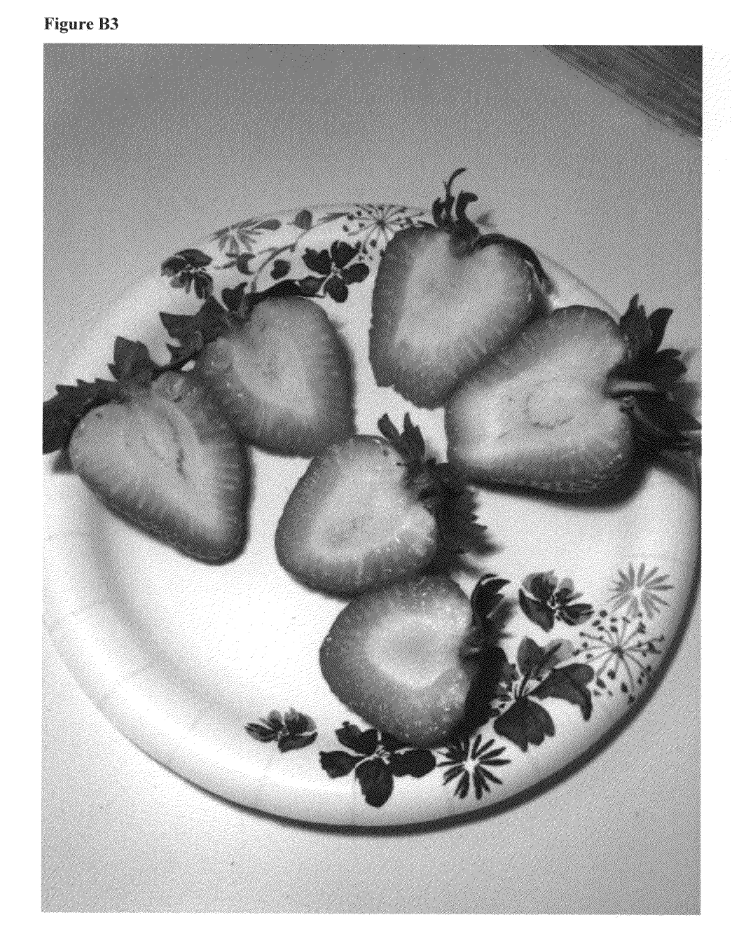

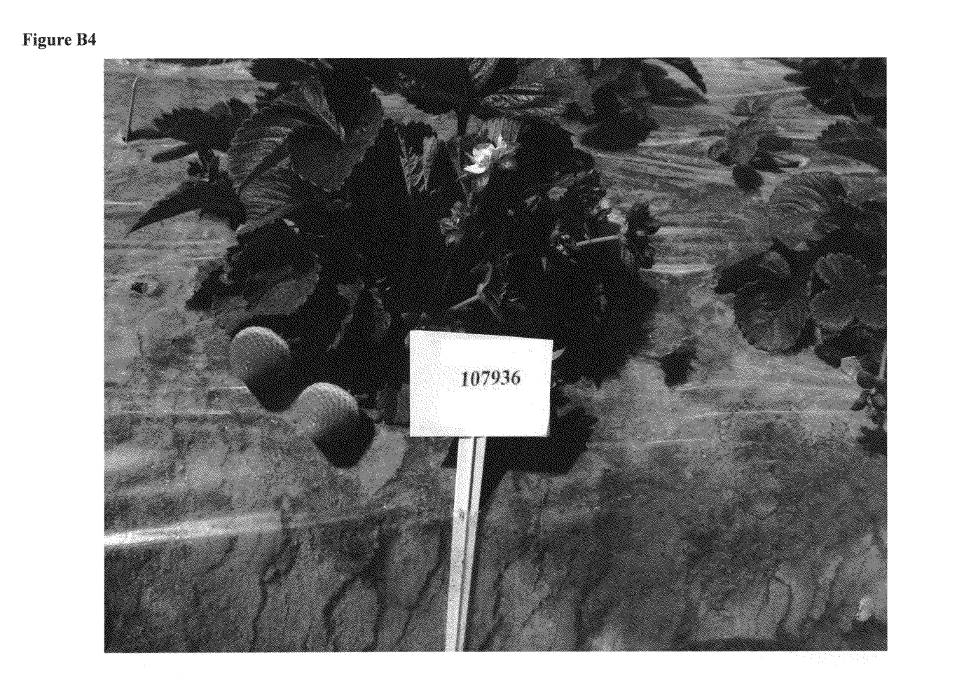

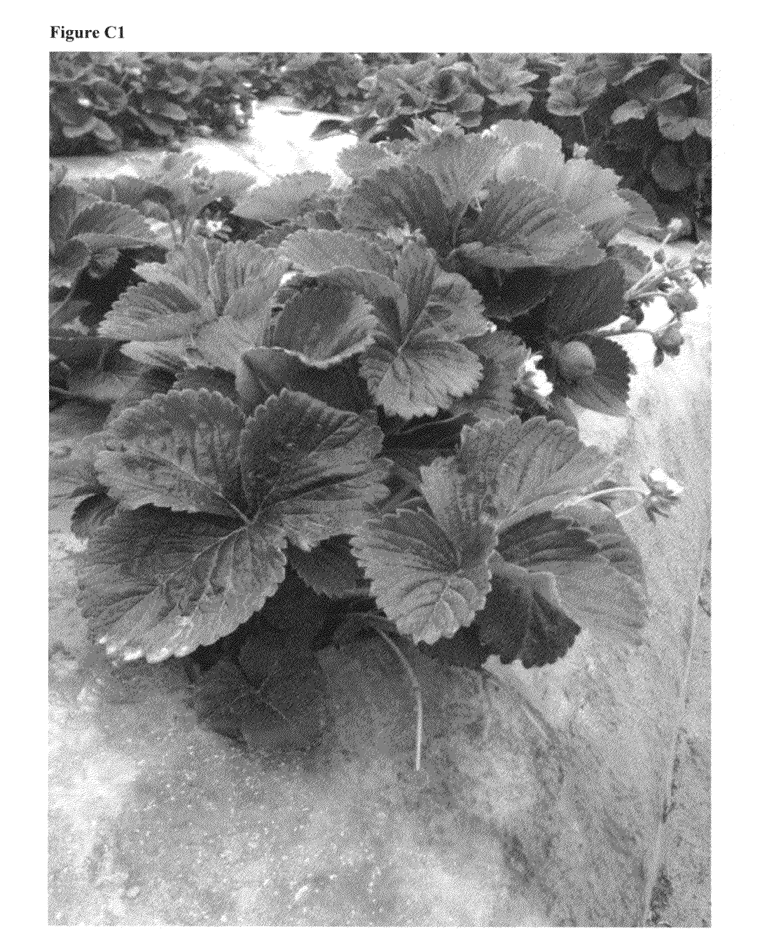

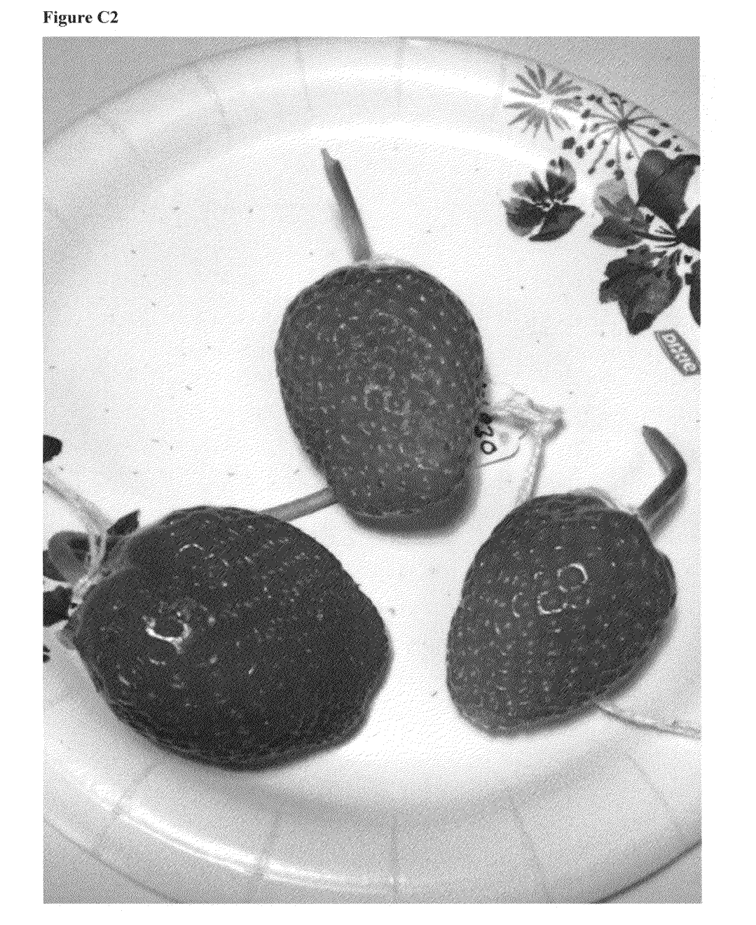

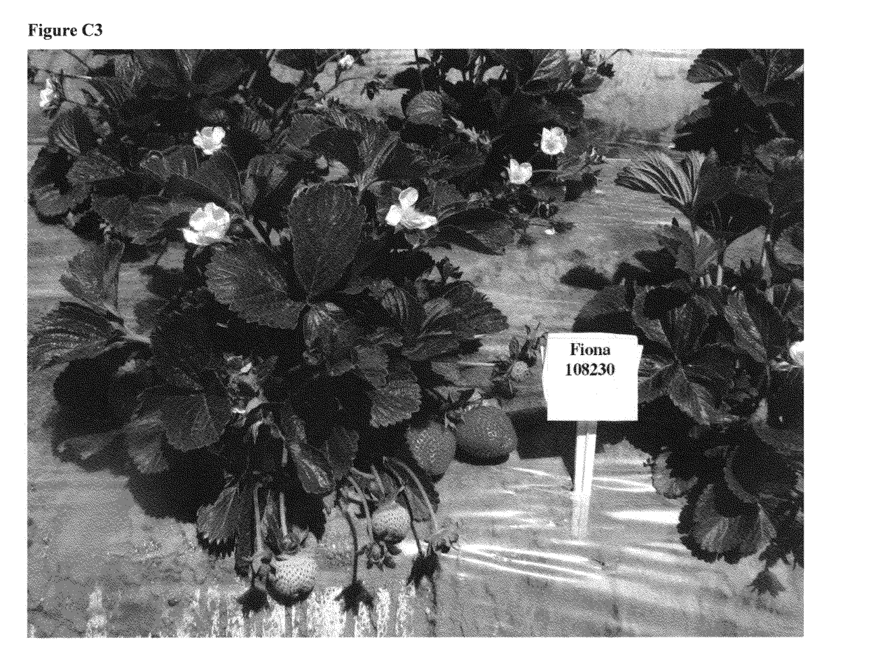

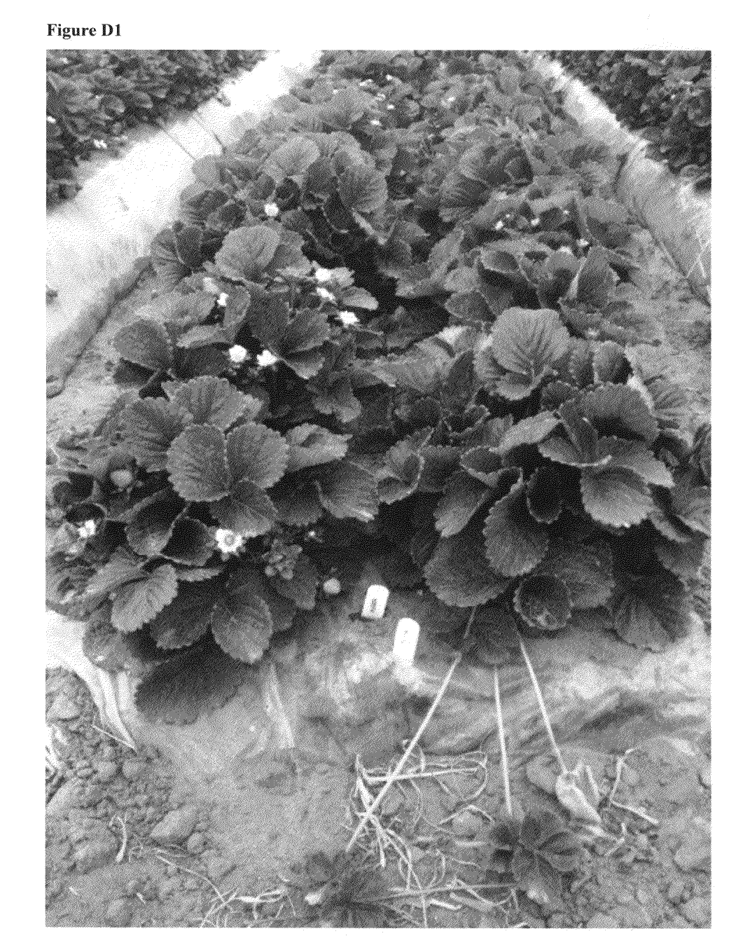

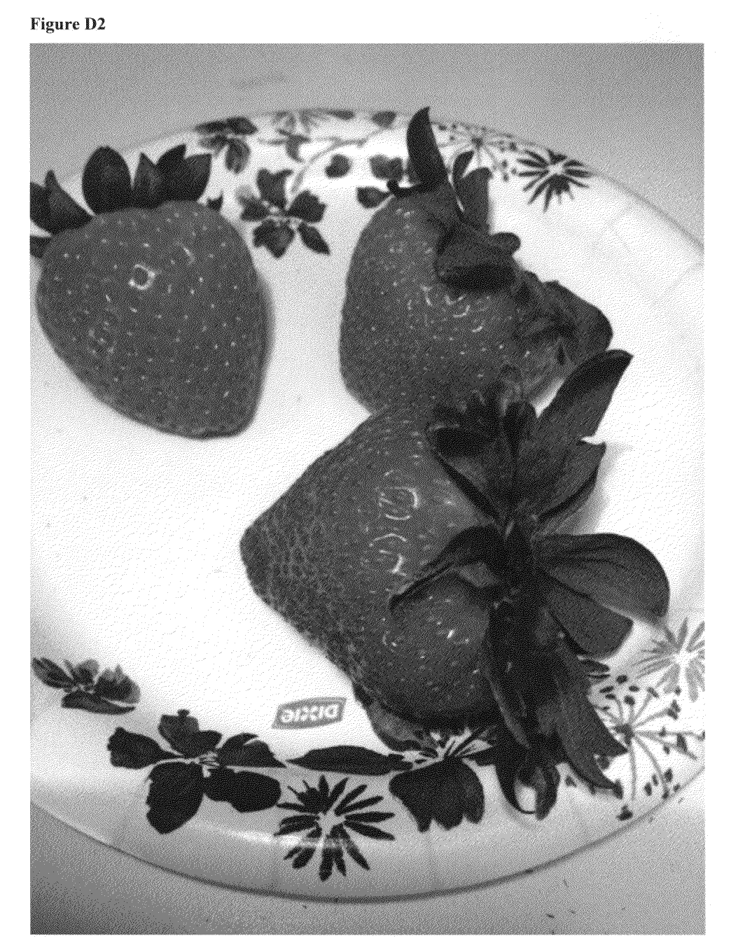

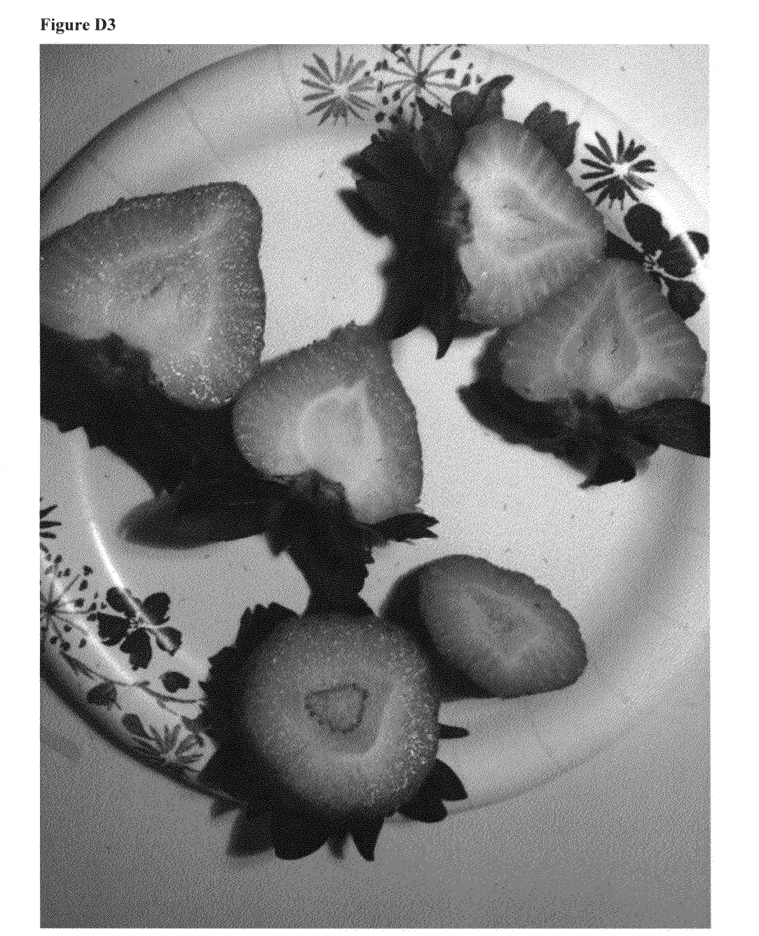

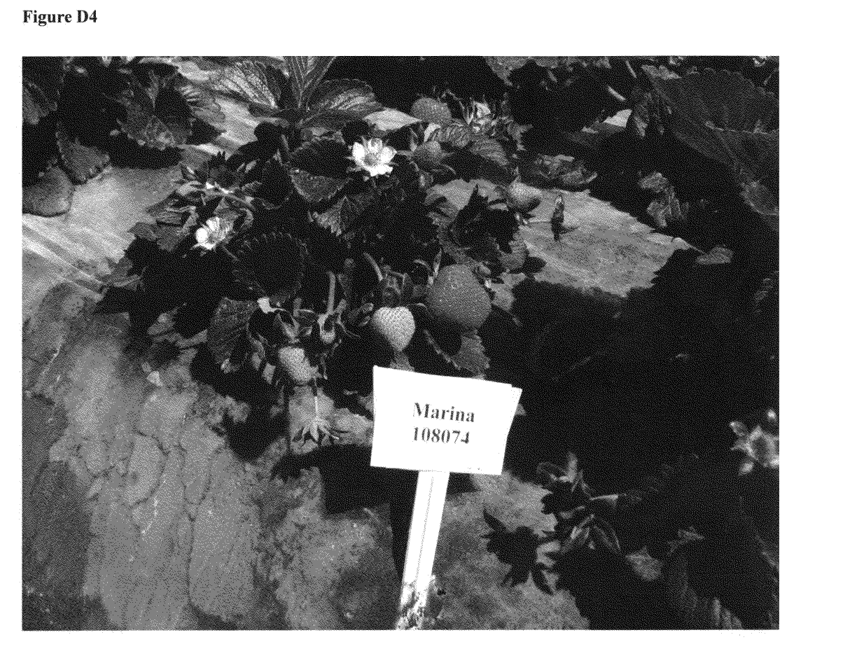

### CROSS-REFERENCE TO RELATED APPLICATIONS

This application claims the benefit of U.S. Provisional Patent Application Ser. No. 61/650,798, filed May 23, 2012, which is hereby incorporated by reference in its entirety for all purposes.

### LATIN NAME OF GENUS AND SPECIES

Fragaria×ananassa

### VARIETAL DENOMINATIONS

‘AURORA’ (a.k.a. ‘107750’)

‘CRYSTALINA’ (a.k.a. ‘107936’, or ‘CRYSTAL’ previously)

‘FIONA’ (a.k.a. ‘108230’)

‘MARINA’ (a.k.a. ‘108074’)

### BACKGROUND OF THE INVENTION

### ‘AURORA’

The present invention relates to a new and distinct early day-neutral strawberry variety designated as ‘AURORA’ (a.k.a. ‘107750’). This new variety is the result of a controlled-cross between a female parent cultivar designated ‘103462’ and a male parent cultivar designated ‘6410’ (both unpatented, proprietary cultivars) made by the inventor and first fruited in Watsonville, Calif. growing fields.

Following selection and during testing, the plant was originally designated ‘107750’ and subsequently named ‘AURORA’. The new variety of ‘AURORA’ was asexually reproduced via runners (stolons) by the inventor at Watsonville, Calif. Asexual propagules from the original source have been tested in the Watsonville growing fields and, to a limited extent, growing fields in low and high elevation. The properties of this variety were found to be transmissible by such asexual reproduction. The cultivar is stable and reproduces true to type in successive generations of asexual reproduction.

### ‘CRYSTALINA’

The present invention also relates to a new and distinct early day-neutral strawberry variety designated as ‘CRYSTALINA’ (a.k.a. ‘107936’, or ‘CRYSTAL’ previously). This new variety is the result of a controlled-cross between a female parent cultivar designated ‘103904’ and a male parent cultivar designated ‘103926’ (both unpatented, proprietary cultivars) made by the inventor and first fruited in Watsonville, Calif. growing fields.

Following selection and during testing, the plant was originally designated ‘107936’ and subsequently named ‘CRYSTALINA’. The new variety of ‘CRYSTALINA’ was asexually reproduced via runners (stolons) by the inventor at Watsonville, Calif. Asexual propagules from the original source have been tested in the Watsonville growing fields and, to a limited extent, growing fields in low and high elevation. The properties of this variety were found to be transmissible by such asexual reproduction. The cultivar is stable and reproduces true to type in successive generations of asexual reproduction.

### ‘FIONA’

The present invention also relates to a new and distinct early day-neutral strawberry variety designated as ‘FIONA’ (a.k.a. ‘108230’). This new variety is the result of a controlled-cross between a female parent cultivar designated ‘102850’ and a male parent cultivar designated ‘106734’ (both unpatented, proprietary cultivars) made by the inventor and first fruited in Watsonville, Calif. growing fields.

Following selection and during testing, the plant was originally designated ‘108230’ and subsequently named ‘FIONA’. The new variety of ‘FIONA’ was asexually reproduced via runners (stolons) by the inventor at Watsonville, Calif. Asexual propagules from the original source have been tested in the Watsonville growing fields and, to a limited extent, growing fields in low and high elevation. The properties of this variety were found to be transmissible by such asexual reproduction. The cultivar is stable and reproduces true to type in successive generations of asexual reproduction.

### ‘MARINA’

The present invention also relates to a new and distinct early day-neutral strawberry variety designated as ‘MARINA’ (a.k.a. ‘108074’). This new variety is the result of a controlled-cross between a female parent cultivar designated ‘104449’ and a male parent cultivar designated ‘101984’ (both unpatented, proprietary cultivars) made by the inventor and first fruited in Watsonville, Calif. growing fields.

Following selection and during testing, the plant was originally designated ‘108074’ and subsequently named ‘MARINA’. The new variety of ‘MARINA’ was asexually reproduced via runners (stolons) by the inventor at Watsonville, Calif. Asexual propagules from the original source have been tested in the Watsonville growing fields and, to a limited extent, growing fields in low and high elevation. The properties of this variety were found to be transmissible by such asexual reproduction. The cultivar is stable and reproduces true to type in successive generations of asexual reproduction.

### SUMMARY OF THE INVENTION

### ‘AURORA’

This invention relates to a new and distinctive early producing day-neutral type cultivar designated as ‘AURORA’. It is primarily adapted to the climate and growing conditions of the central coast of California. This region provides the necessary temperatures required for it to produce a strong vigorous plant and to remain in fruit production from April through October. The nearby Pacific Ocean provides the needed humidity and moderate day temperatures and evening chilling to maintain fruit quality for the production months.

The following traits in combination distinguish strawberry variety ‘AURORA’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

The following are the most outstanding and distinguishing characteristics of this new variety when grown under normal conditions in Watsonville, Calif.

Large fruit; and
High yield

When ‘AURORA’ is compared to the proprietary female parent ‘103462’, ‘AURORA’ is larger in size and has more vigor.

When ‘AURORA’ is compared to the proprietary male parent ‘6410’, ‘AURORA’ produces larger fruit. ‘AURORA’ leaves are significantly larger.

When ‘AURORA’ is compared to ‘Albion’ (patented), ‘AURORA’ produces larger fruit at a slightly later interval. Size is comparable but ‘AURORA’ has larger leaves.

### ‘CRYSTALINA’

This invention also relates to a new and distinctive early producing day-neutral type cultivar designated as ‘CRYSTALINA’. It is primarily adapted to the climate and growing conditions of the central coast of California. This region provides the necessary temperatures required for it to produce a strong vigorous plant and to remain in fruit production from April through October. The nearby Pacific Ocean provides the needed humidity and moderate day temperatures and evening chilling to maintain fruit quality for the production months.

The following traits in combination distinguish strawberry variety ‘CRYSTALINA’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

The following are the most outstanding and distinguishing characteristics of this new variety when grown under normal conditions in Watsonville, Calif.

Large fruit; and
High yield.

When ‘CRYSTALINA’ is compared to the proprietary female parent ‘10394’, ‘CRYSTALINA’ has larger leaflets and larger fruit. ‘CRYSTALINA’ strawberries are more resistant to weather changes and do not bruise as easily as female parent.

When ‘CRYSTALINA’ is compared to the proprietary male parent ‘103926’, ‘CRYSTALINA’ is similar in most aspect save for having higher yield.

When ‘CRYSTALINA’ is compared to ‘Albion’ (U.S. Plant Pat. No. 16,228), ‘CRYSTALINA’ produced hardier plants and a higher yield in berries.

### ‘FIONA’

This invention relates to a new and distinctive early producing day-neutral type cultivar designated as ‘FIONA’. It is primarily adapted to the climate and growing conditions of the central coast of California. This region provides the necessary temperatures required for it to produce a strong vigorous plant and to remain in fruit production from April through October. The nearby Pacific Ocean provides the needed humidity and moderate day temperatures and evening chilling to maintain fruit quality for the production months.

The following traits in combination distinguish strawberry variety ‘FIONA’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

The following are the most outstanding and distinguishing characteristics of this new variety when grown under normal conditions in Watsonville, Calif.

Large fruit; and
High yield.

When ‘FIONA’ is compared to the proprietary female parent ‘102850’, ‘FIONA’ is taller in appearance, produces larger fruit, and is overall hardier.

When ‘FIONA’ is compared to the proprietary male parent ‘106734’, ‘FIONA’ produces a significantly higher yield.

When ‘FIONA’ is compared to ‘Albion’ (patented), ‘FIONA’ is of similar size. However, it has a larger early yield and produces sweet fruit.

### ‘MARINA’

This invention also relates to a new and distinctive early producing day-neutral type cultivar designated as ‘MARINA’. It is primarily adapted to the climate and growing conditions of the central coast of California. This region provides the necessary temperatures required for it to produce a strong vigorous plant and to remain in fruit production from April through October. The nearby Pacific Ocean provides the needed humidity and moderate day temperatures and evening chilling to maintain fruit quality for the production months.

The following traits in combination distinguish strawberry variety ‘MARINA’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

The following are the most outstanding and distinguishing characteristics of this new variety when grown under normal conditions in Watsonville, Calif.

Large fruit; and
High yield

When ‘MARINA’ is compared to the proprietary female parent ‘104449’, ‘MARINA’ produces berries that are more resistant to bruising. It was also observed that ‘MARINA’ has a greater yield.

When ‘MARINA’ is compared to the proprietary male parent ‘101984’, ‘MARINA’ produces larger berries that are more resistant to rain.

When ‘MARINA’ is compared to ‘Albion’ (patented), ‘MARINA’ produces larger strawberries and has a later harvest interval. It is slightly bigger in plant appearance and slightly more vigorous.

### DESCRIPTION OF THE DRAWINGS

The accompanying color photographs depict various characteristics of the cultivars at various stages of development as nearly true as possible to make color reproductions.

FIG. A1 shows ‘AURORA’ early in the picking season.

FIG. A2 shows ‘AURORA’ strawberries from early May picking.

FIG. A3 shows Cross section of early May ‘AURORA’ strawberries.

FIG. A4 shows ripe and near-ripe fruits of ‘AURORA’ in May.

FIG. B1 shows ‘CRYSTALINA’ plant in early May.

FIG. B2 shows ‘CRYSTALINA’ strawberries from early May picking.

FIG. B3 shows Cross section of early May ‘CRYSTALINA’ strawberries.

FIG. B4 shows ripe and near-ripe fruits of ‘CRYSTALINA’ in May.

FIG. C1 shows ‘FIONA’ during early May.

FIG. C2 shows ‘FIONA’ strawberries after picking, calyx removed.

FIG. C3 shows ripe and near-ripe fruits of ‘FIONA’ in May.

FIG. D1 shows ‘MARINA’ plant in early May.

FIG. D2 shows ‘MARINA’ strawberries from early May picking.

FIG. D3 shows Cross section of early May ‘MARINA’ strawberries.

FIG. D4 shows ripe and near-ripe fruits of ‘MARINA’ in May.

### DETAILED BOTANICAL DESCRIPTION

### ‘AURORA’

‘AURORA’ has not been observed under all possible environmental conditions, and the phenotype may vary significantly with variations in environment. The following observations, measurements, and comparisons describe this plant as grown at California, when grown in the field, unless otherwise noted. As stated above, the color determination is in accordance with The Royal Horticultural Society Colour Chart, 1995 Edition, except where general color terms of ordinary dictionary significance are used. Plants for the botanical measurements in the present application are annual plants.

Botanical classification: ‘AURORA’ is a fertile hybrid derived from a cross.
Common name: garden strawberry

### General Description:

Plant habit: moderate, coastal climates

The following description is applied to our plants that are 7 months old as of the time of the measurements.
Classification:
   Species—Fragaria×ananassa.
   Common name.—Garden Strawberry.
   Denomination.—‘AURORA.’
Parentage:
   Female parent.—‘103462’.
   Male parent.—‘6410’.
Plant:
   Height.—13.0 cm.
   Diameter.—22.5 cm.
   Habit.—Globose, upright.
   Density: Medium.
   Vigor.—Strong.
Terminal leaflet:
   Length.—6.4 cm.
   Width.—7.1 mm.
   Length/width ratio.—
   Shape in cross-section.—Concave.
   Blistering.—Low.
   Glossiness.—Medium.
   Average number of leaflets.—Exactly 3.
   Color above.—Dark Green (RHS 137A).
   Color below.—Green (RHS 139C).
   Shape.—Orbicular.
   Margin.—Crenate to Serrate.
   Venation pattern.—Reticulate.
Petiole:
   Length.—8.2 cm.
   Width.—4.7 mm.
   Color.—Yellow Green (RHS 145A).
Petiolule:
   Length.—10.7 cm.
   Width.—2.3 mm.
   Color.—Yellow Green (RHS 145A).
Stolon:
   Average daughters/plant.—About one.
   Diameter.—2.6 mm.
   Color.—Dark Purple Red (RHS 53A).
Inflorescence:
   Position relative to foliage.—At same level.
   Average petals/flower.—5.
   Petal length.—14.2 mm.
   Petal width.—14.3 mm.
   Petal length/width ratio.—0.99.
   Petal shape/base.—Orbicular.
   Petal apex.—Round.
   Petal margin.—Entire.
   Petal spacing.—Slightly overlapping.
   Petal color.—White (RHS 157B).
   Corolla.—32.3 mm.
   Sepal length.—14.3 mm.
   Sepal width.—6.67 mm.
   Sepal length/width ratio.—2.14.
   Sepal color.—Dark Green (RHS 137A).
   Calyx.—36.3 mm (Diameter relative to corolla: Larger).
   Peduncle.—11.4 cm.
   Bract frequency.—Low.
Fruit:
   Fruit truss attitude.—Prostrate.
   Relative size.—Large.
   Fruit length.—42.0 mm.
   Fruit width.—38.7 mm.
   Fruit length/width ratio.—1.09.
   Surface color.—Red (RHS 45A).
   Flesh color.—Orange Red (RHS 41B).
   Core color.—White (RHS 155C).
   Shape.—Predominately conical.
   Average weight/fruit.—20.8 g.
   Average weight/plant.—71.9 g.
   Hollow core length.—24.7 mm.
   Hollow core width.—8.6 mm.
   Hollow core length/width ratio.—2.87.
   Insertion of achenes.—Slightly indented.
   Average achenes/fruit.—261.
   Firmness of flesh.—Medium.
   Glossiness.—Medium.
   Sweetness.—Medium.

### ‘CRYSTALINA’

‘CRYSTALINA’ has not been observed under all possible environmental conditions, and the phenotype may vary significantly with variations in environment. The following observations, measurements, and comparisons describe this plant as grown at California, when grown in the field, unless otherwise noted. As stated above, the color determination is in accordance with The Royal Horticultural Society Colour Chart, 1995 Edition, except where general color terms of ordinary dictionary significance are used. Plants for the botanical measurements in the present application are annual plants.

Botanical classification: ‘CRYSTALINA’ is a fertile hybrid derived from a cross.
Common name: garden strawberry

### General Description:

Plant habit: moderate, coastal climates

The following description is applied to our plants that are 7 months old as of the time of the measurements.
Classification:
   Species.—Fragaria×ananassa.
   Common name.—Garden Strawberry.
   Denomination.—‘CRYSTALINA’.
Parentage:
   Female parent.—‘103904’.
   Male parent.—‘103926’.
Plant:
   Height.—13.6 cm.
   Diameter.—19.4 cm.
   Habit.—Globose, upright.
   Density.—Medium.
   Vigor.—Strong.
Terminal leaflet:
   Length.—6.4 cm.
   Width.—7.4 cm.
   Length/width ratio.—0.87.
   Shape in cross-section.—Concave.
   Blistering.—Low.
   Glossiness.—Medium.
   Average number of leaflets.—Exactly 3.
   Color above.—Dark Green (RHS 137A).
   Color below.—Green (RHS 139C).
   Shape.—Orbicular.
   Margin.—Crenate to Serrate.
   Venation pattern.—Reticulate.
Petiole:
   Length.—7.0 cm.
   Width.—5.1 mm.
   Color.—Yellow Green (RHS 145A).
Petiolule:
   Length.—10.4 cm.
   Width.—2.7 mm.
   Color.—Yellow Green (RHS 145A).
Stolon:
   Average daughters/plant.—About one.
   Diameter.—3.6 mm.
   Color.—Dark Purple Red (RHS 53A).
Inflorescence:
   Position relative to foliage.—At same level.
   Average petals/flower.—6.5.
   Petal length.—15.7 mm.
   Petal width.—15.8 mm.
   Petal length/width ratio.—0.99.
   Petal shape/base.—Orbicular.
   Petal apex.—Round.
   Petal margin.—Entire.
   Petal spacing.—Slightly overlapping.
   Petal color.—White (RHS 157B).
   Corolla.—30.8 mm.
   Sepal length.—20 mm.
   Sepal width.—10.5 mm.
   Sepal length/width ratio.—1.90.
   Sepal color.—Dark Green (RHS 137A).
   Calyx.—53.8 mm (Diameter relative to corolla: Larger).
   Peduncle.—9.0 cm.
   Bract frequency.—Low.
Fruit:
   Fruit truss attitude.—Prostrate.
   Relative size.—Large.
   Fruit length.—40.7 mm.
   Fruit width.—39.1 mm.
   Fruit length/width ratio.—1.04.
   Surface color.—Red (RHS 45A).
   Flesh color.—Orange Red (RHS 41B).
   Core color.—White (RHS 155C).
   Shape.—Predominately conical.
   Average weight/fruit.—22.5 g.
   Average weight/plant.—103.5 g.
   Hollow core length.—30.3 mm.
   Hollow core width.—10.1 mm.
   Hollow core length/width ratio.—3.0.
   Insertion of achenes.—Indented.
   Average achenes/fruit.—213.
   Firmness of flesh.—Medium.
   Glossiness.—Medium.
   Sweetness.—Medium.

### ‘FIONA’

‘FIONA’ has not been observed under all possible environmental conditions, and the phenotype may vary significantly with variations in environment. The following observations, measurements, and comparisons describe this plant as grown at California, when grown in the field, unless otherwise noted. As stated above, the color determination is in accordance with The Royal Horticultural Society Colour Chart, 1995 Edition, except where general color terms of ordinary dictionary significance are used. Plants for the botanical measurements in the present application are annual plants.

Botanical classification: ‘FIONA’ is a fertile hybrid derived from a cross.
Common name: garden strawberry

### General Description:

Plant habit: moderate, coastal climates

The following description is applied to our plants that are 7 months old as of the time of the measurements.
Classification:
   Species.—Fragaria×ananassa.
   Common name.—Garden Strawberry.
   Denomination.—‘FIONA’.
Parentage:
   Female parent.—‘102850’.
   Male parent.—‘106734’.
Plant:
   Height.—17.7 cm.
   Diameter.—24.0 cm.
   Habit.—Globose, upright.
   Density.—Medium.
   Vigor.—Strong.
Terminal leaflet:
   Length.—7.4 cm.
   Width.—7.3 cm.
   Length/width ratio.—1.02.
   Shape in cross-section.—Concave.
   Blistering.—Low.
   Glossiness.—Medium.
   Average number of leaflets.—Exactly 3.
   Color above.—Dark Green (RHS 137A).
   Color below.—Green (RHS 139C).
   Shape.—Orbicular.
   Margin.—Crenate to Serrate.
   Venation pattern.—Reticulate.
Petiole:
   Length.—11 cm.
   Width.—4.8 mm.
   Color.—Yellow Green (RHS 145A).
Petiolule:
   Length.—5.7 mm.
   Width.—2.5 mm.
   Color.—Yellow Green (RHS 145A).
Stolon:
   Average daughters/plant.—About one.
   Diameter.—3.7 mm.
   Color.—Dark Purple Red (RHS 53A).
Inflorescence:
   Position relative to foliage.—At same level.
   Average petals/flower.—5.
   Petal length.—18.1 mm.
   Petal width.—17.2 mm.
   Petal length/width ratio.—1.05.
   Petal shape/base.—Orbicular.
   Petal apex.—Round.
   Petal margin.—Entire.
   Petal spacing.—Slightly overlapping.
   Petal color.—White (RHS 157B).
   Corolla.—38.2 mm.
   Sepal length.—15.3 mm.
   Sepal width.—7.0 mm.
   Sepal length/width ratio.—2.19.
   Sepal color.—Dark Green (RHS 137A).
   Calyx.—39.2 mm (Diameter relative to corolla: Larger).
   Peduncle.—12.3 cm.
   Bract frequency.—Low.
Fruit:
   Fruit truss attitude.—Prostrate.
   Fruit/truss.—About three.
   Relative size.—Large.
   Fruit length.—39.4 mm.
   Fruit width.—38.0 mm.
   Fruit length/width ratio.—1.04.
   Surface color.—Red (RHS 45A).
   Flesh color.—Orange Red (RHS 41B).
   Core color.—White (RHS 155C).
   Shape.—Predominately conical.
   Average weight/fruit.—19.0 g.
   Average weight/plant.—110.6 g.
   Hollow core length.—21.57 mm.
   Hollow core width.—8.6 mm.
   Hollow core length/width ratio.—2.51.
   Insertion of achenes.—Indented.
   Average achenes/fruit.—237.
   Firmness of flesh.—Medium.
   Glossiness.—Medium.
   Sweetness.—Medium.

### ‘MARINA’

‘MARINA’ has not been observed under all possible environmental conditions, and the phenotype may vary significantly with variations in environment. The following observations, measurements, and comparisons describe this plant as grown at California, when grown in the field, unless otherwise noted. As stated above, the color determination is in accordance with The Royal Horticultural Society Colour Chart, 1995 Edition, except where general color terms of ordinary dictionary significance are used. Plants for the botanical measurements in the present application are annual plants.

Botanical classification: ‘MARINA’ is a fertile hybrid derived from a cross.
Common name: garden strawberry

### General Description:

Plant habit: moderate, coastal climates

The following description is applied to our plants that are 7 months old as of the time of the measurements.
Classification:
   Species: Fragaria×ananassa.
   Common name.—Garden Strawberry.
   Denomination.—‘MARINA’.
Parentage:
   Female parent.—‘104449’.
   Male parent.—‘101984’.
Plant:
   Height.—15.6 cm.
   Diameter.—24.2 cm.
   Habit.—Globose, upright.
   Density.—Medium.
   Vigor.—Strong.
Terminal leaflet:
   Length.—7.3 cm.
   Width.—9.2 cm.
   Length/width ratio.—0.79.
   Shape in cross-section.—Concave.
   Blistering.—Low.
   Glossiness.—Medium.
   Average number of leaflets.—Exactly 3.
   Color above.—Dark Green (RHS 137A).
   Color below.—Green (RHS 139C).
   Shape.—Orbicular.
   Margin.—Crenate to Serrate.
   Venation pattern.—Reticulate.
Petiole:
   Length.—7.9 cm.
   Width.—6.0 mm.
   Color.—Yellow Green (RHS 145A).
Petiolule:
   Length.—7.0 mm.
   Width.—2.3 mm.
   Color.—Yellow Green (RHS 145A).
Stolon:
   Average daughters/plant.—About one.
   Diameter.—3.3 mm.
   Color.—Dark Purple Red (RHS 53A).
Inflorescence:
   Position relative to foliage.—At same level.
   Average petals/flower.—5.5.
   Petal length.—14.3 mm.
   Petal width.—14.3 mm.
   Petal length/width ratio.—1.00.
   Petal shape/base.—Orbicular.
   Petal apex.—Round.
   Petal margin.—Entire.
   Petal spacing.—Slightly overlapping.
   Petal color.—White (RHS 157B).
   Corolla.—33.1.
   Sepal length.—14.0 mm.
   Sepal width.—8.3 mm.
   Sepal length/width ratio.—1.70.
   Sepal color.—Dark Green (RHS 137A).
   Calyx.—37.0 mm(Diameter relative to corolla: Larger).
   Peduncle.—8.8 cm.
   Bract frequency.—Low.
Fruit:
   Fruit truss attitude.—Prostrate.
   Relative size.—Large.
   Fruit length.—37.5 mm.
   Fruit width.—39.2 mm.
   Fruit length/width ratio.—0.96.
   Surface color.—Red (RHS 45A).
   Flesh color.—Orange Red (RHS 41B).
   Core color.—White (RHS 155C).
   Shape.—Predominately wedge.
   Average weight/fruit.—18.9 g.
   Average weight/plant.—78.1 g.
   Hollow core length.—16.4 mm.
   Hollow core width.—7.1 mm.
   Hollow core length/width ratio.—2.31.
   Insertion of achenes.—Indented.
   Average achenes/fruit.—242.
   Firmness offlesh.—Medium.
   Glossiness.—Medium.
   Sweetness.—Medium.

1. A new and distinct cultivar of strawberry plant named ‘AURORA’, ‘CRYSTALINA’, ‘FIONA’, or ‘MARINA’ substantially as shown and described herein.
